(19) United States
(12) Patent Application Publication
(10) Pub. No.: US 2004/0116888 A1
(43) Pub. Date: Jun. 17, 2004

(54) Absorbent article having increased extension of extensible inner and outer facings during wear

(76) Inventor: Linda Dorschner, Kaukauna, WI (US)

Correspondence Address: PAULEY PETERSEN KINNE & ERICKSON, 2800 WEST HIGGINS ROAD, SUITE 365, HOFFMAN ESTATES, IL, 60195, US

(21) Appl. No.: 10/319,084

(22) Filed: Dec. 13, 2002

(51) Int. Cl. A61F 13/15; A61F 13/20

(52) U.S. Cl. 604/385.22

(57) ABSTRACT

A disposable diaper pant having laterally opposing side seams connecting a front waist region of the absorbent article to a back waist region of the absorbent article, and forming a chassis. The chassis having a percent extension at a waist region of at least about 40% in the cross-machine direction as measured between the side seams. Each side seam bonding an edge portion of the front waist region including a first portion of an extensible outer cover and an edge portion of the back waist region including a second portion of the extensible outer cover. An extensible fastener is bonded at each side seam.

### FIELD OF INVENTION

The present invention relates to disposable absorbent articles. More particularly, the present invention relates to an absorbent article, such as a diaper pant, having extensible inner and outer facing layers, wherein a front waist region and a back waist region of a diaper pant are bonded together at laterally opposing side seams to provide an increased fit or size range.

### BACKGROUND OF THE INVENTION

Absorbent articles such as diapers, diaper pants, training pants or incontinence garments typically include a liner material, an absorbent core, and a liquid impervious back sheet or outer cover. Such articles desirably provide a close, comfortable fit about the wearer and contain body exudates. Diaper pants (articles that are applied like pants but that have the absorbent capacity of a regular diaper), for example, may provide a fastening system for adjustment of the diaper pant about the waist of the wearer, wherein a fastener is bonded to the back region of the diaper pant. Referring to FIG. 1, diaper pants generally include a back ear panel that extends laterally from the back region of the diaper pant to which the fastener is permanently attached or bonded. The ear panel and/or fastener typically comprise a stretchable material, such as a neck-bonded laminate material, and a fastening component, such as adhesive tape tab or a hook-type component. To adjust the fit of the diaper pant on the wearer, the fastening components are releasably engageable with an attachment component, such as a loop-type fastening material positioned on the outer surface of the front waist region. Generally, the loop-type fastening material is made of an inelastic or non-extensible material, such as a point unbonded (PUB) material.

For example, diaper pants may include a loop-type fastening material that extends laterally across a front portion of the diaper pant (and extends laterally beyond the longitudinal side edges of the chassis) and is bonded directly to the back ear panel to form a waist opening. Laterally opposing portions of the fastener material extend laterally from the front edge portions of the diaper to form front ear portions that are typically bonded to the back ear panel using conventional ultrasonic bonding techniques and/or patterns.

During the manufacturing process, registration of the front ear portions to the corresponding back ear panel, in the lateral or cross-machine direction and the longitudinal or machine direction may be difficult and may result in misalignment of the diaper components. Misalignment of the diaper components may result in a diaper that does not provide adequate coverage and leakage protection during use. For example, it can be difficult to register the stretchable back ear panel with the corresponding inelastic or non-extensible front ear portion during the bonding process to bond the two materials together to form the waist opening, without misalignment or misregistration of the materials. Additionally, it may be difficult to align the front waist region with the back waist region longitudinally or in the machine direction to properly form the waist opening.

Another potential disadvantage associated with bonding extensions of the loop material in the front waist region to stretchable back ear panels is that additional materials may be required. For example, when the loop material is passively bonded to the back ear panel, additional nonessential or unused material that is relatively expensive, such as the neck-bonded laminate material forming the ear panel and/or the pattern unbonded material forming the attachment component may be required. In addition, the excess material makes it difficult to fold the back ear panel with respect to the corresponding front ear portion in order to fasten the mechanical fastener (e.g. a hook material) to the attachment component in the front waist region of the diaper.

Additionally, many diapers and diaper pants include leg elastics having a high curvature corresponding to the curvature of the leg openings formed in a carrier sheet during the manufacturing process. However, highly curved leg elastics can be difficult to apply to the carrier sheet during the manufacturing process. Typically, such highly curved leg elastics include strands of elastic material, for example, that are applied in a curved orientation on the carrier sheet with respect to the leg openings formed by the carrier sheet. The elastic strands are typically applied to the carrier sheet using adhesive bonding materials. The elastic strands may tend to move or roll on the carrier sheet with respect to the leg openings during the subsequent manufacturing of the diaper, resulting in misalignment of the leg elastics, thus potentially defeating the purpose of the leg elastics.

In addition to the manufacturing difficulties described above, conventional diapers and diaper pants do not always provide adequate coverage about a wearer's waist, which ultimately results in undesired leakage. For example, the inelastic or non-extensible loop material bonded to the back ear panel may limit the extensibility of other diaper components that may have been selected to be extensible. Therefore, when a caregiver attempts to fasten or reposition the fasteners of the diaper pant about the wearer, the stretchable portion of the back ear panel stretches but the other components of the diaper forming the chassis, including the front portion and the back portion, may not be able to extend to conform to or to cover the wearer's body. For example, the chassis may not properly stretch or extend to cover the wearer's buttocks and hips and/or provide a seal about the wearer's legs. Thus, the diaper pants may not provide adequate coverage and/or seal, which can undesirably result in improper fit and/or leakage. As a result, the diaper pants may not contain bodily exudates as effectively as desired.

Accordingly, despite the attempts to develop diaper pants and other absorbent articles, there remains a need for a diaper pant having an extensible chassis that is capable of conforming to the wearer for proper fit and effective containment of bodily exudates. More specifically, there remains a need for a diaper pant for which the pant-forming seam/bond does not interfere with the extension properties of the chassis materials.

Further, there remains a need for simplifying and/or improving the manufacturing processes of diaper pants, thereby improving lateral and longitudinal component registration during the manufacturing process while minimizing the use of relatively expensive component materials, such as neck-bonded laminate materials and pattern unbonded materials, as well as simplifying the addition of components, such as leg elastics.

### SUMMARY OF THE INVENTION

In response to the difficulties and problems discussed above, the present invention provides a prefastened diaper pant wherein a back waist region of a chassis including extensible outer and inner facing layers is bonded to a front waist region of a chassis including extensible outer and inner facing layers at laterally opposing side seams to form a chassis having a waist opening and two leg openings. A stretchable or extensible fastener is positioned between and bonded to the edge portions of the front waist region and the back waist region forming the side seam. Thus, as the fastener is pulled to adjust the fit of the diaper pant about the waist of the wearer, the extensible outer and inner facing layers are also pulled to properly cover the wearer's buttocks and hips to provide a seal about the wearer's legs, for example.

The diaper pant of the present invention includes an extensible outer cover and an extensible bodyside liner that is connected to the outer cover in a superposed relation. An absorbent core is located between the outer cover and the bodyside liner to define a chassis of the diaper pant that forms the waist opening and each leg opening.

Desirably, the outer cover and/or the bodyside liner comprising the front waist region and the back waist region are extensible in the cross-machine direction to allow the chassis to extend beyond its original manufactured dimensions at the waist opening during wear. Because the outer cover and/or the bodyside liner are extensible, a percent extension of the chassis in the waist area of the diaper pant is increased. Further, a partial side seam or a full side seam may be used to connect the front waist region to the back waist region in order to increase the extensibility of the front and back waist regions.

In one embodiment of this invention, the diaper pant of the present invention has a percent extension about the waist opening of at least about 40%, more desirably at least about 60%, and most desirably at least about 70%. The percent extension of the diaper pant at the waist opening can be defined as a difference between an extended or final waist measurement (“Wₑ”) and a relaxed or initial waist measurement (“Wᵢ”) divided by the initial waist measurement, multiplied by 100, wherein We is a lateral measurement of a width of the back waist region of the diaper pant between the laterally opposing side seams, when the diaper pant is properly positioned on a wearer, and Wᵢ is a lateral measurement of the width of the back waist region of the diaper pant between the laterally opposing side seams, in a relaxed configuration. The lateral direction of the diaper pant is the direction generally parallel to the waistline of the diaper pant. The longitudinal direction is the direction generally perpendicular to the waistline, from the front waist region to the back waist region of the diaper pant, through the crotch region.

The configuration of the diaper pant of the present invention also provides several manufacturing and/or processing advantages and simplifications. For example, directly bonding an ear portion of the front waist region of the chassis including extensible outer and inner facing layers to the corresponding ear portion of the back waist region of the chassis including extensible outer and inner facing layers results in improved and simplified registration of the diaper pant components in the lateral direction as well as in the longitudinal direction. Such direct bonding of the front waist region ear to the corresponding back waist region ear eliminates the need for expensive additional materials as used in conventional diaper pants, such as the pattern unbonded material forming the front ears and the elastic neck-bonded laminate materials forming portions of the back ear panels of conventional diaper pants. Further, the diaper pant of the present invention can include straight leg elastics rather than curvilinear leg elastics, thus further simplifying the manufacturing process, for example, and overcoming problems associated with conventional manufacturing processes. Because the leg elastics are straight, the elastic or extensible materials forming the straight leg elastics do not move or roll with respect to the leg openings during manufacturing.

### BRIEF DESCRIPTION OF THE DRAWINGS

These and other objects and features of this invention will be better understood from the following detailed description taken in conjunction with the drawings and examples, wherein:

FIG. 1 representatively shows a perspective view of a conventional disposable diaper pant having a prefastened configuration;

FIG. 2 representatively shows a perspective view of a disposable diaper pant, according to one embodiment of this invention;

FIG. 3 representatively shows a front plan view of the disposable diaper pant of FIG. 2, according to one embodiment of this invention;

FIG. 4 representatively shows a front plan view of the disposable diaper pant of FIG. 2 with a waist size adjustment means in a disengaged position, according to one embodiment of this invention;

FIG. 5 representatively shows a plan view of the disposable diaper pant of FIG. 2 in an unfastened, stretched and laid flat condition with a surface of the diaper pant that contacts the wearer's skin facing the viewer, according to one embodiment of this invention;

FIG. 6 representatively shows a plan view of the disposable diaper pant of FIG. 2 in an unfastened, stretched and laid flat condition with a surface of the diaper pant that contacts the wearer's skin facing away from the viewer, according to one embodiment of this invention;

FIG. 7 displays the load (Grams) versus the extension (mm) for Sample 1 of Example 2, according to one embodiment of this invention;

FIG. 8 displays the load (Grams) versus the extension (mm) for Samples 2-11 of Example 2; and

FIG. 9 representatively shows a plan view of the Cross-Directional Measurement performed in Example 3 below.

### DETAILED DESCRIPTION OF THE INVENTION DEFINITIONS

As used herein, the term “disposable” refers to articles that are intended to be discarded after a limited use and that are not intended to be laundered or otherwise restored for reuse.

As used herein, the terms “elastic,” “elasticized” and “elasticity” refer to a property of a material or composite by virtue of which the material or composite tends to recover its original size and shape after removal of a force causing a deformation.

As used herein, the term “elastomeric” refers to a material or composite which can be elongated by at least 50 percent of its relaxed length in any direction and which will recover, upon release of the applied force, at least 40 percent of its elongation. It is generally desired that the elastomeric material or composite be capable of being elongated by at least 100 percent, more desirably by at least 300 percent, of its relaxed length and recover, upon release of an applied force, at least 50 percent of its elongation.

As used herein, the term “extensible” refers to a material or composite which can be elongated by at least 50% of its relaxed length in any direction and tends not to recover, or recovers less than 50% of its elongation, desirably less than 30% of its elongation, after removal of a force causing a deformation.

As used herein, the term “inelastic” refers to materials that are not elastomeric, either because they cannot be sufficiently stretched by the above amount or because they do not sufficiently retract by the above amount when stretched and relaxed.

As used herein, the terms “necked” and “neck stretched” are interchangeable terms that refer to a method of elongating an inelastic nonwoven fabric, generally in the longitudinal, or machine direction of the fabric, to reduce its width in a controlled manner to a desired amount. The controlled stretching may take place under cool, room temperature, or greater temperatures, and is limited to an increase in overall dimension in the direction being stretched up to the elongation required to break the fabric, which in most cases is about 1.2 to 1.4 times an original length. The resulting neck-stretched fabric can be extended in the lateral (cross-machine) direction of the fabric during subsequent use, causing the fabric to return toward its original pre-necked configuration. Neck stretching processes are disclosed, for example, in U.S. Pat. No. 4,443,513 to Meitner et al.; U.S. Pat. No. 4,965,122, U.S. Pat. No. 4,981,747 and U.S. Pat. No. 5,114,781 to Morman; and U.S. Pat. No. 5,244,482 to Hassenboehler Jr. et al.

As used herein, the term “passive bond” refers to a bond which has a relatively low peel strength such that the bond can be easily broken by hand if desired to assist in inspecting or removing an absorbent article from the wearer, without tearing or severely damaging the other portions of the article, and without causing trauma to the wearer or spillage of waste material from the absorbent article.

As used herein “pattern unbonded” or interchangeably “point unbonded” or “PUB,” means a fabric pattern having continuous bonded areas defining a plurality of discrete unbonded areas. The fibers or filaments within the discrete unbonded areas are dimensionally stabilized by the continuous bonded areas that encircle or surround each unbonded area, such that no support or backing layer of film or adhesive is required. The unbonded areas are specifically designed to afford spaces between fibers or filaments within the unbonded areas. A suitable process for forming the pattern-unbonded nonwoven material of this invention includes providing a nonwoven fabric or web, providing opposedly positioned first and second calender rolls and defining a nip therebetween, with at least one of said rolls being heated and having a bonding pattern on its outermost surface comprising a continuous pattern of land areas defining a plurality of discrete openings, apertures or holes, and passing the nonwoven fabric or web within the nip formed by said rolls. Each of the openings in said roll or rolls defined by the continuous land areas forms a discrete unbonded area in at least one surface of the nonwoven fabric or web in which the fibers or filaments of the web are substantially or completely unbonded. Stated alternatively, the continuous pattern of land areas in said roll or rolls forms a continuous pattern of bonded areas that define a plurality of discrete unbonded areas on at least one surface of said nonwoven fabric or web. Alternative embodiments of the aforesaid process include pre-bonding the nonwoven fabric or web before passing the fabric or web within the nip formed by the calender rolls, or providing multiple nonwoven webs to form a pattern-unbonded laminate. PUB fabrics are disclosed in U.S. patent application Ser. No. 08/754,419, commonly assigned, the disclosure of which is incorporated herein by reference.

Alternative applications in which PUB fabric may be used include those having film applied during the formation of the PUB fabric where the film will provide a liquid barrier so that it may be used as a clothlike outer cover for a personal care product.

As used herein, the term “permanently bonded” refers to the joining, adhering, connecting, attaching, or the like, of two elements of an absorbent article such that the elements tend to be and remain bonded during normal use conditions of the absorbent article.

As used herein, the term “prefastened” refers to a condition wherein the absorbent article has a fastening feature, which is engaged or fastened prior to use by the wearer. For example, the fastening feature of the absorbent article may be engaged or fastened during the manufacturing process.

As used herein, the term “refastenable” refers to the property of two elements being capable of releasable attachment, separation, and subsequent releasable reattachment without substantial permanent deformation or other damage to either element.

As used herein, the term “releasably attached,” “releasably engaged,” and variations thereof refer to two elements being connected or connectable such that the elements tend to remain connected absent a separation force applied to one or both of the elements and the elements being capable of separation without substantial permanent deformation or other damage. The required separation force is typically beyond that encountered while wearing the absorbent article.

The various aspects and embodiments of the present invention will be described in the context of disposable absorbent articles, and more particularly referred to, without limitation and by way of illustration only, as a disposable diaper pant. It is apparent that the articles and methods of the present invention are equally adaptable for other types of absorbent articles, such as training pants, adult incontinence articles, feminine care articles, other personal care garments, medical or health care garments, and other disposable articles and garments.

The diaper pant 20 of the present invention suitably has a percent extension at the waist opening of at least about 40%, desirably at least about 60% and in many cases at least about 70%. The percent extension (“E”) of the diaper pant 20 typically is defined as a difference between an extended or final waist measurement (“Wₑ”) and a relaxed or initial waist measurement (“Wᵣ”) divided by the initial waist measurement, multiplied by 100, as set forth in the following equation:

E=[(Wₑ−Wᵣ)/Wᵣ]×100; Eq. (1)

wherein Wᵣ is a lateral measurement of a width of the back waist region of the diaper pant 20, between laterally opposing side seams in a relaxed configuration and Wₑ is a lateral measurement of the width of the back waist region of the diaper pant 20 between laterally opposing side seams, when the diaper pant 20 is properly positioned on a wearer, for example.

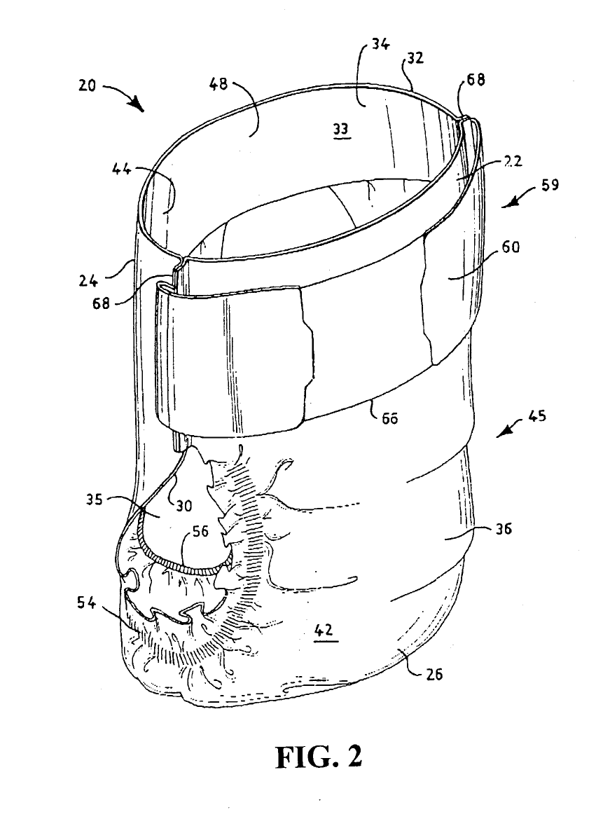

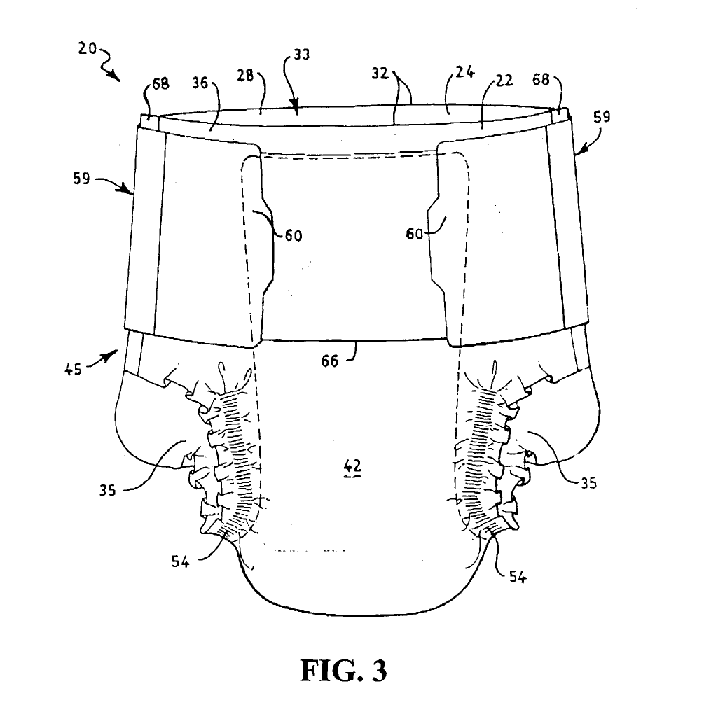

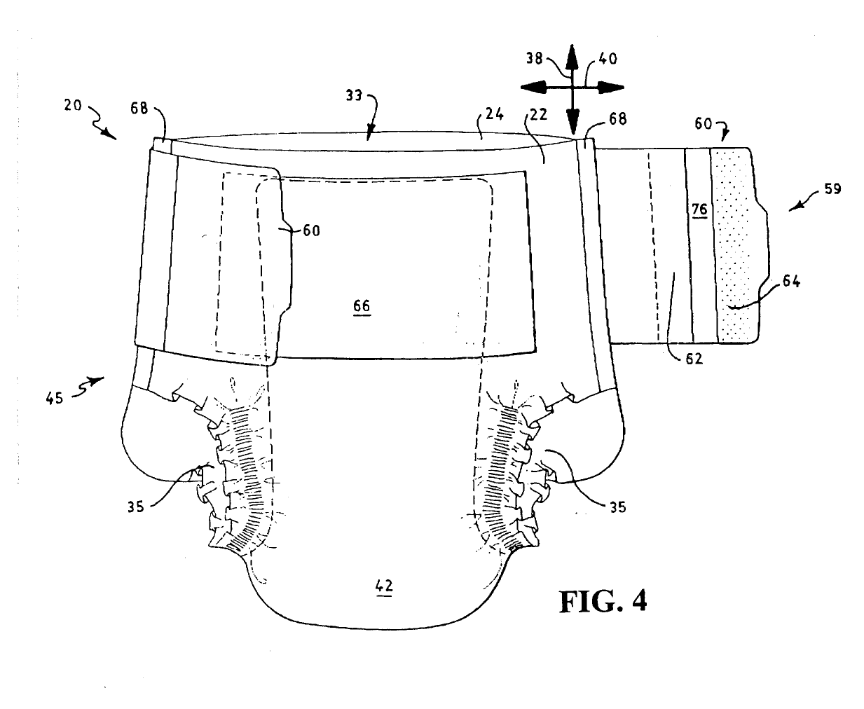

Referring to FIGS. 2-6, the disposable prefastened diaper pant 20 of the present invention defines a front waist region 22, a back waist region 24, and an intermediate or crotch region 26 that extends between and connects the front waist region 22 and the back waist region 24. For reference, arrows 38 and 40 depicting the orientation of the longitudinal axis and the lateral axis, respectively, of the diaper pant 20 are illustrated in FIGS. 4-6.

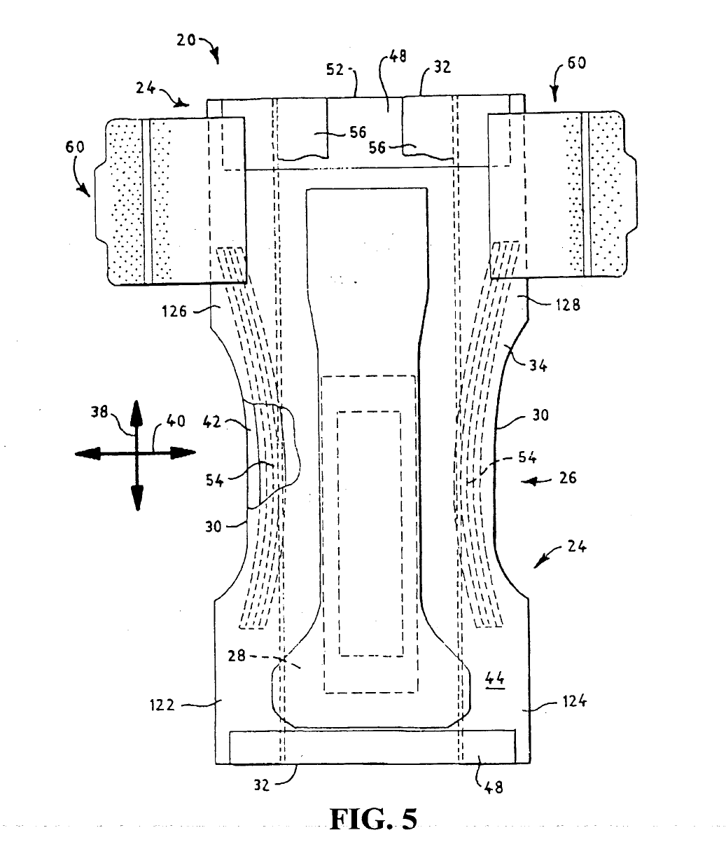

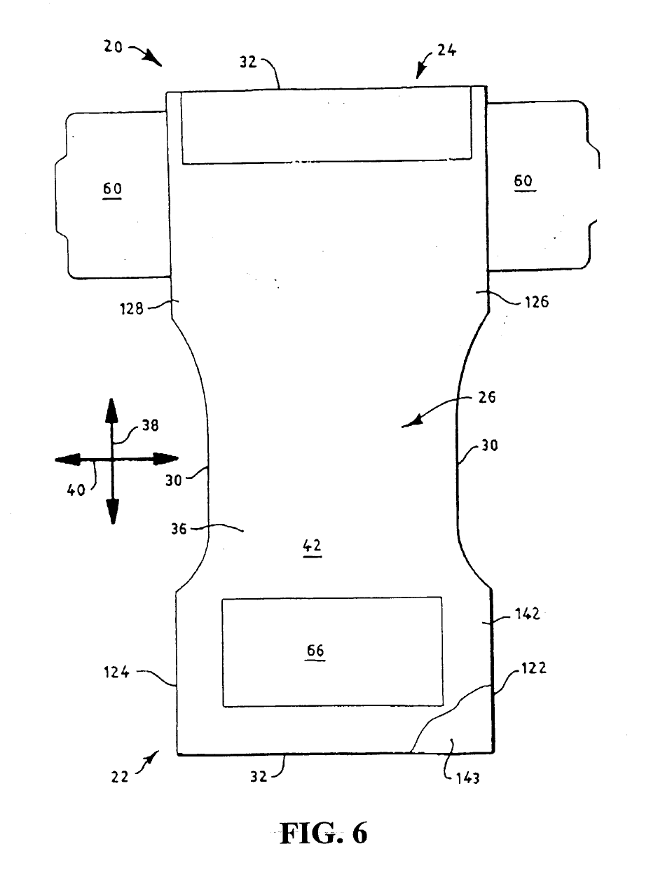

As shown in FIGS. 5 and 6, the front waist region 22 includes the portion of the diaper pant 20 that, when worn, is positioned on the front of the wearer while the back waist region 24 includes the portion of the diaper pant 20 that, when worn, is positioned on the back of the wearer. The crotch region 26 of the diaper pant 20 includes the portion of the diaper pant 20 that, when worn, is positioned between the legs of the wearer and covers the lower torso of the wearer.

The diaper pant 20 defines a pair of laterally opposing side edges 30, a pair of longitudinally opposing waist edges 32, an interior surface 34 (FIG. 5) that is configured to contact the wearer, and an exterior surface 36 (FIG. 6) opposite the interior surface 34 that is configured to contact the wearer's clothing during use. Referring to FIGS. 2-6, the diaper pant 20 also includes an outer cover 42 and a bodyside liner 44 that is connected to the outer cover 42 in a superposed relation. An absorbent core 28 is located between the outer cover 42 and the bodyside liner 44. The outer cover 42, the bodyside liner 44 and the absorbent core 28 define a chassis 45 of the diaper pant 20, which forms a waist opening 33 and each leg opening 35. The laterally opposing side edges 30 are generally defined by the side edges of the outer cover 42 and/or the bodyside liner 44 that further define leg openings 35. As shown in FIG. 5, desirably, but not necessarily, laterally opposing side edges 30 are generally parallel in the longitudinal direction 38 of the diaper pant 20, which results in easier processability. Alternatively, in certain embodiments of this invention, the laterally opposing side edges 30 may desirably be curvilinear. The waist edges 32 are generally defined by the waist edges of the outer cover 42 and/or the bodyside liner 44 and form the waist opening 33 that is configured to encircle the waist of the wearer during use. The absorbent core 28 is configured to contain and/or absorb body exudates discharged from the wearer.

The diaper pant 20 may be of various suitable shapes. For example, referring to the unfastened configuration as shown in FIGS. 5 and 6, the diaper pant 20 may have an overall rectangular shape, T-shape or an approximate hourglass shape. In the shown embodiment, the diaper pant 20 has a generally I-shape in an unfastened configuration. Examples of diaper configurations suitable for use in connection with the instant application and other diaper components suitable for use on diapers are described in U.S. Pat. No. 4,798,603 issued Jan. 17, 1989 to Meyer et al.; U.S. Pat. No. 5,176,668 issued Jan. 5, 1993 to Bernardin; U.S. Pat. No. 5,192,606 issued Mar. 9, 1993 to Proxmire et al.; and U.S. Pat. No. 5,509,915 issued Apr. 23, 1996 to Hanson et al., the disclosures of which are herein incorporated by reference.

The various components of the diaper pant 20 are integrally assembled together employing various types of suitable attachment means, such as adhesive, sonic, and thermal bonds, and combinations thereof. In the shown embodiment, for example, the outer cover 42 and bodyside liner 44 may be assembled to each other and to the absorbent core 28 with an adhesive, such as a hot melt, pressure-sensitive adhesive. The adhesive may be applied as a uniform continuous layer of adhesive, a patterned layer of adhesive, a sprayed pattern of adhesive, or an array of separate lines, swirls or dots of adhesive. Similarly, the other components of the diaper pant 20 may be assembled into the diaper pant 20 employing suitable attachment mechanisms.

In accordance with one embodiment of this invention, as shown in FIGS. 2-6, the outer cover 42 is extensible, for example as described in U.S. patent application Ser. No. 09/563,417 filed on May 3, 2000 by Roessler et al. The disclosure of application Ser. No. 09/563,417 is incorporated herein by reference to the extent it assists the present disclosure. Desirably, the extensible outer cover 42 is extensible in at least a lateral direction, generally parallel to the lateral axis represented by arrow 40 in FIG. 6. The outer cover 42 of the diaper pant 20 may suitably be composed of a material, which is either water vapor permeable or water vapor impermeable. It is generally desired that the outer cover 42 be formed from a material that is substantially impermeable to liquids and permeable (breathable) to water vapor. The outer cover 42 can be manufactured from a thin plastic film or other suitable flexible liquid-impermeable material. For example, the outer cover 42 may be formed from a polyethylene film having a thickness of about 0.013 millimeter (0.5 mil) to about 0.051 millimeter (2.0 mils). Desirably, for a more clothlike feeling, the outer cover 42 may be formed from a polyolefin film having a nonwoven web, such as a spunbond web of polyolefin fibers, laminated to an exterior surface thereof. For example, a stretch-thinned polypropylene film having a thickness of about 0.015 millimeter (0.6 mil) may have thermally laminated thereto a spunbond web of polypropylene fibers. The polypropylene fibers have a thickness of about 1.5 to 2.5 denier per filament, which nonwoven web has a basis weight of about 17 grams per square meter (0.5 ounce per square yard). The outer cover 42 may otherwise include bicomponent fibers such as polyethylene/polypropylene bicomponent fibers. Methods of forming such clothlike outer covers are known to those skilled in the art.

For example, in one embodiment of this invention, the outer cover 42 comprises a dual composite material including an outer facing layer 142 and an inner facing layer 143 at least partially bonded or laminated to the outer facing layer 142, as shown in FIG. 6. Desirably, both the outer facing layer 142 and the inner facing layer 143 are extensible in at least the lateral or cross-machine direction, as represented by arrow 40. In one embodiment of this invention, the extensible outer facing layer 142 comprises a spunbond nonwoven material and the extensible inner facing layer 143 comprises a film material laminated to the outer facing layer 142. The outer facing layer 142 and/or the inner facing layer 143 may comprise any other suitable extensible material.

Further, the outer cover 42 may be formed of a woven or nonwoven fibrous web layer which has been totally or partially constructed or treated to impart a desired level of liquid impermeability to selected regions that are adjacent or proximate the absorbent core 28. Still further, the outer cover 42 may optionally be composed of a microporous breathable material that permits vapors to escape from the absorbent core 28 while still preventing liquid exudates from passing through the outer cover 42. For example, the outer cover 42 may include a vapor permeable nonwoven outer facing layer 142 laminated to an inner facing layer 143 comprising a microporous film. Suitable breathable outer cover materials are described in U.S. Pat. No. 5,695,868, issued to McCormack et al.; and U.S. Pat. No. 5,843,056, issued Dec. 1, 1998 to Good et al., the disclosures of which are incorporated herein by reference. Still further, the outer cover 42 may also be an elastomeric material such as a stretch-thermal laminate (STL), a neck-bonded laminate (NBL), or a stretch-bonded laminate (SBL) material. Methods of making such materials are well known to those skilled in the art and are described in U.S. Pat. No. 4,663,220, issued May 5, 1987 to Wisneski et al.; U.S. Pat. No. 5,226,992, issued Jul. 13, 1993 to Morman; and European Patent Application No. EP 0 217 032 published on Apr. 8, 1987 in the names of Taylor et al., the disclosures of which are incorporated herein by reference. The outer cover 42 can also be embossed or otherwise provided with a matte finish to provide a more aesthetically pleasing appearance.

As shown in FIG. 5, the bodyside liner 44 suitably presents a bodyfacing surface that is compliant, soft feeling, and nonirritating to the wearer's skin. In accordance with one embodiment of this invention, the bodyside liner 44 is extensible. For example, the bodyside liner 44 may be made from extensible materials as are described in U.S. patent application Ser. No. 09/563,417 filed on May 3, 2000 by Roessler et al. Desirably, the bodyside liner 44 is extensible in at least the lateral direction.

Further, the bodyside liner 44 may be less hydrophilic than the absorbent core 28 to present a relatively dry surface to the wearer and may be sufficiently porous to be liquid permeable, permitting liquid to readily penetrate through its thickness. Suitable web materials for manufacturing the bodyside liner 44 include, but are not limited to, porous foams, reticulated foams, apertured plastic films, natural fibers (for example, wood or cotton fibers), synthetic fibers (for example, polyester or polypropylene fibers), and/or a combination of natural and synthetic fibers. The bodyside liner 44 is suitably employed to help isolate the wearer's skin from liquids held in the absorbent core 28.

Various woven and nonwoven fabrics can be used for the bodyside liner 44. For example, the bodyside liner 44 may be composed of a meltblown or spunbond web of polyolefin fibers. The bodyside liner 44 may also be a bonded-carded web composed of natural and/or synthetic fibers. The bodyside liner 44 may be composed of a substantially hydrophobic material, and the hydrophobic material may optionally be treated with a surfactant or otherwise processed to impart a desired level of wettability and hydrophilicity. In one embodiment of the invention, the bodyside liner 44 is made from a nonwoven, spunbond polypropylene fabric composed of about 2.8 to about 3.2 denier fibers formed into a web having a basis weight of about 20 grams per square meter and a density of about 0.13 grams per cubic centimeter. The fabric may be surface treated with about 0.3 weight percent of a surfactant commercially available from Hodgson Textile Chemicals, Inc. under the trade designation AHCOVEL Base N-62. The surfactant may be applied by any conventional means, such as spraying, printing, brush coating or the like. The surfactant may be applied to the entire bodyside liner 44 or may be selectively applied to particular sections of the bodyside liner 44, such as the medial section along the longitudinal centerline of the diaper pant, to provide greater wettability of such sections. The bodyside liner 44 may further include a lotion or treatment applied thereto that is configured to be transferred to the wearer's skin.

The absorbent core 28 of the diaper pant 20 may suitably be composed of a matrix of hydrophilic fibers, such as a web of cellulosic fluff, mixed with particles of a high-absorbency material commonly known as superabsorbent material. In one embodiment of this invention, the absorbent core 28 includes a matrix of cellulosic fluff such as wood pulp fluff and superabsorbent hydrogel-forming particles. The wood pulp fluff may be exchanged with synthetic, polymeric, meltblown fibers, or with a combination of meltblown fibers and natural fibers. The superabsorbent particles may be substantially homogeneously mixed with the hydrophilic fibers or may be nonuniformly mixed. The fluff and superabsorbent particles may also be selectively placed into desired zones of the absorbent core 28 to better contain and absorb body exudates. The concentration of the superabsorbent particles may also vary through the thickness of the absorbent core 28. Alternatively, the absorbent core 28 may comprise a laminate of fibrous webs and superabsorbent material or other suitable means of maintaining a superabsorbent material in a localized area.

Suitable high-absorbency materials for the absorbent core 28 include, but are not limited to, natural, synthetic, and modified natural polymers and materials. The high-absorbency materials can be inorganic materials, such as silica gels, or organic compounds, such as crosslinked polymers. The term “crosslinked” refers to any means for effectively rendering normally water-soluble materials substantially water insoluble but swellable. Such means can include, for example, physical entanglement, crystalline domains, covalent bonds, ionic complexes and associations, hydrophilic associations such as hydrogen bonding, and hydrophobic associations or Van der Waals forces.

Examples of suitable synthetic, polymeric, high-absorbency materials include, but are not limited to, the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrolidone), poly(vinyl morpholinone), poly(vinyl alcohol), and mixtures and copolymers thereof. Further polymers suitable for use in the absorbent core 28 include, but are not limited to, natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthan gum, locust bean gum, and similar compounds. Mixtures of natural and wholly or partially synthetic absorbent polymers can also be useful in the present invention. Such high-absorbency materials are well known to those skilled in the art and are widely commercially available. Examples of superabsorbent polymers suitable for use in the present invention are SANWET IM 3900 polymer available from Hoechst Celanese located in Portsmouth, Va. and DOW DRYTECH 2035LD polymer available from Dow Chemical Co. located in Midland, Mich.

The high absorbency material may be in any of a wide variety of geometric forms. Generally, it is desired that the high absorbency material be in the form of discrete particles. However, the high absorbency material may also be in the form of fibers, flakes, rods, spheres, needles, or the like. Generally, the high absorbency material is present in the absorbent core 28 in an amount of about 5 weight percent to about 90 weight percent, based on a total weight of the absorbent core 28.

In accordance with one embodiment of this invention, a substantially hydrophilic tissue wrapsheet (not shown) may be employed to help maintain the integrity of the airlaid fibrous structure of the absorbent core 28. The tissue wrapsheet is typically placed about the absorbent core 28 over at least one of the major facing surfaces thereof and composed of an absorbent cellulosic material, such as creped wadding or a high wet-strength tissue. In accordance with one embodiment of the invention, the tissue wrapsheet can be configured to provide a wicking layer that helps to rapidly distribute liquid over the mass of absorbent fibers comprising the absorbent core 28. The tissue wrapsheet on one side of the absorbent fibrous mass may be bonded to the tissue wrapsheet located on the opposite side of the fibrous mass to effectively entrap the absorbent core 28. In accordance with one embodiment of this invention, the tissue wrapsheet may be necked, pleated and/or have any suitable design known to those having ordinary skill in the art to extend in at least one direction, for example the lateral or cross-machine direction, without tearing or ripping.

In accordance with one embodiment of this invention, as shown in FIGS. 2 and 5, for example, the diaper pant 20 includes a pair of containment flaps 56 that are configured to provide a barrier to the lateral flow of body exudates. The containment flaps 56 may be located along the laterally opposing side edges 30 of the diaper pant 20 adjacent the side edges of the absorbent core 28. Each containment flap 56 typically defines an unattached edge that is configured to maintain an upright, perpendicular configuration in at least the crotch region 26 of the diaper pant 20 to form a seal against the wearer's body. Each containment flap 56 extends longitudinally along at least a portion of a length of the absorbent core 28. Desirably, each containment flap 56 extends along substantially the entire length of the absorbent core 28 to better contain the body exudates. In accordance with one embodiment of this invention wherein each containment flap 56 extends along a portion of the length of the absorbent core 28, the containment flaps 56 can be selectively positioned along the side edges 30 of the diaper pant 20 in the crotch region 26. Such containment flaps 56 are generally well known to those skilled in the art. For example, suitable constructions and arrangements for containment flaps 56 are described in U.S. Pat. No. 4,704,96, issued Nov. 3, 1987 to K. Enloe, the disclosure of which is hereby incorporated by reference.

In accordance with one embodiment of this invention, at least a portion of the waist edges 32 and the side edges 30 are elastic or extensible to improve the percent extension of the diaper pant 20 and support the absorbent core 28 to prevent leakage of body exudates. For example, as shown in FIGS. 2-5, the diaper pant 20 may include a pair of leg elastic members 54 that are connected to the laterally opposing side edges 30 of the diaper pant 20 in the crotch region 26. The leg elastic members 54 are generally adapted to fit about the legs of a wearer to maintain a positive, contacting relationship with the wearer to effectively reduce or eliminate the leakage of body exudates from the diaper pant 20.

Materials suitable for use as the leg elastic members 54 are well known to those skilled in the art. For example, sheets, filaments, strands or ribbons of a polymeric, elastomeric material may be adhered to the outer cover 42 in a stretched position or attached to the outer cover 42 while the outer cover 42 is pleated, such that elastic constrictive forces are imparted to the outer cover 42. The leg elastic members 54 may also include such materials as polyurethane, synthetic rubber and natural rubber.

In accordance with one embodiment of this invention as shown in FIG. 5, the leg elastic members 54 may be applied to the outer cover 42 during the diaper pant manufacturing process in a generally straight orientation along the longitudinal or machine direction 38. Desirably, but not necessarily, the leg elastic members 54 are applied or bonded to the outer cover 42 using adhesive bonding techniques. For example, an adhesive spray may be used to bond the leg elastic members 54 to the outer cover 42. By applying the leg elastic members 54 in a straight orientation, the manufacturing process is simplified and the misalignment of the leg elastic members 54 with respect to the corresponding leg opening 35 is eliminated or reduced. Further, because the diaper pant chassis according to one embodiment of this invention includes an extensible outer cover 42 and/or extensible bodyside liner 44 that makes the diaper pant chassis generally extensible in the lateral or cross-machine direction, as the diaper pant 20 is pulled in the lateral or cross-machine direction to fit about the waist of the wearer, the leg elastic members 54 are stretched as the diaper pant chassis is stretched to form leg elastic members 54 having a curvature to properly fit about the legs of the wearer. Thus, the straight leg elastic members 54 of the present invention act to form a seal about the wearer's legs as conventional leg elastic members having a high curvature, without the processing problems associated with conventional highly curved leg elastic members.

In accordance with one embodiment of this invention as shown in FIG. 5, the diaper pant 20 includes a fit panel 48 superimposed adjacent to the waist edge 32 in at least one of the front waist region 22 and the back waist region 24 to provide a more comfortable, contouring fit about the wearer. Desirably, the fit panel 48 is elastic, elastomeric or extensible in at least one direction, such as the cross-machine or lateral direction. For example, as shown in FIG. 5, the diaper pant 20 includes an elastomeric fit panel 48 on the interior surface 34 of the diaper pant 20 that is configured to elongate in the lateral direction to provide an improved percent extension and enhanced appearance of the diaper pant 20. Desirably, the elastic or extensible fit panel 48 allows the waist opening 33 to be adjusted to properly fit the diaper pant 20 about the waist of the wearer. The fit panel 48 is also configured with respect to the diaper pant 20 such that the absorbent core 28 has the ability to move and receive body exudates without adversely affecting the positioning of the fit panel 48 and the diaper pant 20 about the waist of the wearer. Thus, movements of the wearer may cause the absorbent core 28 to move but do not adversely affect the overall positioning and fit of the diaper pant 20 on the wearer. Such improved fit can result in reduced leakage from the diaper pant 20, increased comfort, and a more aesthetically pleasing appearance. The fit panel 48 may be attached to the diaper pant 20 in any suitable manner that provides the desired elastic, elastomeric or extensible properties. For example, the fit panel 48 may be attached to the diaper pant 20 using adhesive, ultrasonic, and/or thermal bonding techniques, and the like.

As shown in FIG. 5, in accordance with one embodiment of this invention, the fit panel 48 is located on the interior surface 34 and extends longitudinally beyond the side edges of the absorbent core 28 so that the fit panel 48 is generally coterminous with the waist edge 32 in the respective front waist region 22 and/or back waist region 24.

Suitable materials for producing the fit panel 48 include, but are not limited to, stretch-bonded laminate (SBL) materials, neck-bonded laminate (NBL) materials, elastomeric films, elastomeric foam materials, and/or the like. For example, suitable meltblown elastomeric fibrous webs are described in U.S. Pat. No. 4,663,220, issued May 5, 1987 to T. Wisneski et al., the disclosure of which has previously been incorporated herein by reference. Examples of suitable composite fabrics comprising at least one layer of a nonwoven material secured to a fibrous elastic layer are described in European Patent Application No. EP 090 010 published on Apr. 8, 1987 with the inventors listed as J. Taylor et al., the disclosure of which has previously been incorporated by reference. Examples of suitable NBL materials are described in U.S. Pat. No. 5,226,992, issued Jul. 13, 1993 to Morman, the disclosure of which has previously been incorporated herein by reference.

In accordance with one embodiment of this invention as shown in FIGS. 2-4, the front waist region 22 and the back waist region 24 are at least partially bonded or attached together at laterally opposing side edges 30 of the chassis 45 by side seams 68. Side seams 68 are desirably formed using ultrasonic bonding means well known in the art. Alternatively, or in addition to the ultrasonic bonding means, other suitable bonding means known in the art, such as adhesive bonding means, may be used to bond the front waist region 22 to the rear waist region 24 at each side seam 68. Desirably, but not necessarily, each side seam 68 extends from the waist opening 33 to the corresponding leg opening 35. In one embodiment of this invention, at least one of the side seams 68 may extend partially between the waist opening 33 and the corresponding leg opening 35 to form an “intermittent” or “partial” side seam 68.

Referring to FIGS. 2-4, the side seam 68 desirably bonds the outer cover 42 and the bodyside liner 44 at the side edge 30 of the front waist region 22 to the outer cover 42 and bodyside liner 44 at the corresponding side edge 30 of the back waist region 24. As discussed above, desirably the outer cover 42 comprises an extensible dual composite material, wherein the extensible outer facing layer 142 comprises a suitable nonwoven material, such as a spunbond nonwoven material, and the extensible inner facing layer 143 comprises a suitable film material laminated to the nonwoven material.

In one embodiment of this invention, the extensible front waist region 22 includes a first portion of the extensible outer cover 42 and a first portion of the extensible bodyside liner 44. The front waist region forms a first front ear or edge portion 122 and a laterally opposing second front ear or edge portion 124, as shown in FIGS. 5 and 6. The extensible back waist region 24 includes a second portion of the outer cover 42 and the bodyside liner 44 and forms a first back ear or edge portion 126 and a laterally opposing second back ear or edge portion 128, as shown in FIGS. 5 and 6. The first side seam 68 bonds the first front ear portion 122 to the first back ear portion 126 and the second side seam 68, laterally opposing the first side seam 68, bonds the second front ear portion 124 to the second back ear portion 128. As a result of bonding the front ear portions 122 and 124 to the back ear portions 126 and 128, respectively, the chassis 45 at the waist opening 33 is capable of having a suitable percent extension of at least about 40%, desirably the percent extension is at least about 60% and in many cases, the percent extension is at least about 80%.

Additionally, directly bonding the front ear portions 122 and 124 to the back ear portions 126 and 128, respectively, results in improved registration of the diaper pant components in the lateral or cross-machine direction and in the longitudinal or machine-direction, thus, simplifying the manufacturing process. For example, the processing difficulties associated with conventional absorbent articles discussed above are reduced or eliminated. Additionally, the need for expensive materials, as required in conventional manufacturing processes, including neck-bonded laminate materials and PUB materials is eliminated or minimized.

The side seams 68 are configured to maintain the diaper pant 20 in a prefastened configuration as it is pulled on and off over the hips of the wearer during use. Thus, it is desirable that the side seams 68 provide adequate strength to maintain the front ears aligned without twisting or folding while donning. Such strengths are well known to those skilled in the art.

Referring to FIGS. 2-4, in accordance with one embodiment of this invention, the diaper pant 20 includes a fastening system 59 having at least one, and desirably two laterally opposing refastenable fasteners 60. Referring further to FIGS. 5 and 6, the fasteners 60 are permanently bonded, adhered or otherwise attached directly to the diaper pant 20 at or laterally inward from its side edges 30, in one of the front ear portions 122 and 124 or the back ear portions 126 and 128. The fasteners 60 may be permanently bonded or attached in the vicinity of the side edges 30 of the diaper pant 20 by any means known to those skilled in the art, such as adhesive bonds, sonic bonds and/or thermal bonds. Desirably, the fasteners 60 are permanently bonded or attached to the back waist region 24, as shown in FIG. 5.

In accordance with one embodiment of this invention as shown in FIG. 4, each fastener 60 may include an extensible portion 62 and a waist size adjustment means, for example a fastening portion 64. The extensible portion 62 may be permanently bonded or attached to the side edge 30 in one of the front waist region 22 and/or the back waist region 24. Desirably, the extensible portion 62 is positioned between and attached or bonded to the side edge 30 of the front waist region 22 and the corresponding side edge 30 of the back waist region 24 when the side seam 68 is formed, for example by ultrasonic bonding means. Suitable materials for the extensible portion 62 include, but are not limited to, stretch-bonded laminate (SBL) materials, neck-bonded laminate (NBL) materials, elastomeric films, elastomeric foam materials, laminates of nonwoven webs with elastomeric strands, filaments or films, and the like, such as described above as being suitable for the fit panel 48. Desirably, the extensible portion 62 comprises a neck-bonded laminate material or a stretch-bonded laminate material.

Attaching the fasteners 60 directly onto the front waist region 22 or the back waist region 24 or directly to the extensible fit panel 48, provides the improved fit range and coverage of the present invention. For example, in one embodiment of this invention, fasteners 60 are positioned between and bonded to the front waist region 22 and the back waist region 26 at corresponding side seam 68. Because the extensible portion 62 of fastener 60 is bonded to the extensible outer cover 42 at side seam 68, as the fastener 60 is disengaged from the attachment panel 66 (discussed below), for example, and pulled to reposition the diaper pant 20 about the waist of the wearer, the extensible outer cover 42 is pulled or extended with the fastener 60 to provide proper coverage about the wearer's buttocks and hips. Thus, the diaper pant 20 of the present invention provides added fit, flexibility, and an optimum seal in the corresponding front waist region 22 and the back waist region 24 once the diaper pant 20 is positioned on the wearer.

At least a portion of each fastener 60, for example the fastening portion 64, can be releasably engagable directly with the outer surface 36 of the diaper pant 20 to provide improved fit and ease of fastening. Alternatively, as shown in FIGS. 2-4 and 6, the diaper pant 20 can include an attachment panel 66 located on the outer cover 42 in at least one of the front waist region 22 and the back waist region 24 of the diaper pant 20 to which each fastener 60 is refastenably engageable to maintain the diaper pant 20 about the waist of the wearer. The attachment panel 66 may include a single piece of material that extends across at least a portion of the respective waist region of the diaper pant 20, as shown in FIGS. 2-4 and 6. Desirably, but not necessarily, the attachment panel 66 comprises a pattern unbonded (PUB) material or any other suitable loop-type material releasably engageable with the fastening portion 64 of the fastener 60.

The fastening portion 64 extends laterally from the side seam 68 to form the waist size adjustment means. The fastening portions 64 of the fasteners 60 are configured to encircle the hips of the wearer and engage the attachment panel 66 or the exterior surface 36 of the front waist region 22 of the diaper pant 20 to reduce the waist perimeter dimension of the waist opening 33 and conform the waist opening 33 to the wearer's waist. The waist perimeter dimension is a peripheral or circumferential measurement of the waist opening 33, which should generally correspond to the waist of the wearer during use of the diaper pant 20. It is apparent that the fastening portion 64 may alternatively be located on the front waist region 22 and may be configured to releasably engage an attachment panel 66 positioned on the back waist region 24 or the exterior surface 36 of the back waist region 24. Alternatively, the diaper pant 20 may include a single fastening portion 64 extending from one of the side seams 68.

As shown in FIG. 4, the fasteners 60 may include an intermediate portion 76 between the extensible portion 62 and the fastening portion 64. For example, when the fastener 60 includes hook-type fastener elements on the fastening portion 64, the intermediate portion 76 may be devoid of hook-type fastener elements. Desirably, the intermediate portion 76 is made of an inelastic or non-extensible material.

In one embodiment of this invention, the intermediate portion 76 of the fastener 60 may include a hinge to which the extensible portion 62 and the fastening portion 64 are attached. When the intermediate portion 76 is configured as a hinge, the fastener 60 desirably is thinner at the intermediate portion 76, and therefore more flexible for easier attachment of the fastening portion 64 to the attachment panel 66 or the exterior surface 36 of the diaper pant 20.

Desirably, the fastening portion 64 is made of a suitable releasably engageable fastener, such as an adhesive tape tab fastener, hook fastener, loop fastener, mushroom fastener, snap, pin, belt and the like, and combinations thereof. For example, as shown in FIG. 4, the fastening portion 64 may include a plurality of hook-type fasteners and the attachment panel 66 and/or the outer cover 42 may be configured to function as a complimentary loop-type fastener.

After the initial donning of the diaper pant 20, at least one of the refastenable fasteners 60 provides the waist size adjustment means for adjusting the waist perimeter dimension of the waist opening 33, as shown in FIG. 2. With the diaper pant 20 positioned about the waist of the wearer, the waist perimeter dimension may be adjusted by disengaging or unfastening at least one of the fasteners 60 from the attachment panel 66 and reengaging or refastening the at least one fastener 60 at a lateral position along the attachment panel 66 to either reduce or increase the waist perimeter dimension corresponding to the waist opening 33. Thus, it is desirable that the waist size adjustment means define a relatively low peel strength such that the caregiver can readily disengage the waist size adjustment means from the diaper pant 20. Suitable peel strength values are well known to those skilled in the art. One test for measuring peel strength of hook and loop fasteners is ASTM D1876-72. This test is described in U.S. Pat. No. 5,176,671, issued to Roessler et al., the disclosure of which is incorporated herein by reference.

In accordance with one embodiment of this invention, the fasteners 60 are refastenably engaged with the attachment panel 66 positioned on the outer surface 36 of the front waist region 22 before the diaper pant 20 is placed on the wearer to provide a disposable refastenable, prefastened diaper pant 20. In such a configuration, the diaper pant 20 can be pulled on or off over the legs and hips of the wearer. If the diaper pant 20 becomes soiled during use, the fasteners 60 can be disengaged to easily remove the diaper pant 20 from the waist of the wearer with reduced risk of undesirably soiling the clothes or legs of the wearer. Thus, the diaper pant 20 is configured to be pulled on or off over the hips of the wearer such as conventional training pants and can be readily applied or removed by disengaging the fasteners 60, similar to conventional diaper pant articles. Moreover, the fasteners 60 can be repositioned after initial donning if necessary to adjust the fit of the diaper pant 20 to the wearer.

Alternatively, the fasteners 60 can be folded such that the fasteners 60 are releasably engaged with the outer surface 36 of the back waist region 24 or an attachment panel 66 positioned on the outer surface 36 of the back waist region before the diaper pant 20 is placed on the wearer. In such a configuration, the diaper pant 20 can be pulled over the legs and hips of the wearer without the fasteners 60 engaging other components of the diaper pant 20 or any surrounding material that makes it difficult to apply the diaper pant 20. After the initial donning of the diaper pant 20, the fasteners 60 can be disengaged from the back waist region 24 and engaged with the attachment panel 66 or the outer surface 36 of the front waist region 22 to adjust the waist perimeter dimension to properly fit the waist of the wearer.

The diaper pant 20 of the present invention wherein the front ear portions 122 and 124 are directly bonded to the back ear portions 126 and 128, respectively, results in improved fit. For example, if the caregiver pulls the fasteners 60 to adjust the fit about the wearer's waist, the extensible back waist region 24, as well as the extensible front waist region 22, is extended in the cross-machine direction to provide proper coverage of the wearer's buttocks and hips. In comparison, when a conventional diaper pant, having an attachment component made of an inelastic or non-extensible material bonded to a stretchable ear panel, is stretched, the ear panel stretches to allow attachment of the fastener to the attachment component. However, the back waist region and the front waist region do not stretch or extend in the cross-machine direction to provide proper coverage of the wearer's buttocks and hips.

Further, as the extensible waist regions 22, 24 of the diaper pant 20 of present invention are pulled, the straight leg elastics 54 are stretched as the diaper pant chassis is stretched, to conform to the curvature of the wearer's legs, thereby providing an adequate seal about the wearer's legs.

### EXAMPLE 1

Five samples (Samples 1-5) of a disposable diaper pant 20 of the present invention were made by bonding a front edge portion of the diaper pant 20 including an extensible outer cover 42 and an extensible bodyside liner 44 to a back edge portion of the diaper pant 20 including the extensible outer cover 42 and the extensible bodyside liner 44 at laterally opposing side seams 68 to form a chassis 45 forming a waist opening 33. An extensible portion 62 of a fastener 60 was bonded between the front edge portion and the back edge portion at each side seam 68. The extensible portion 62 comprises a neck-bonded laminate material.

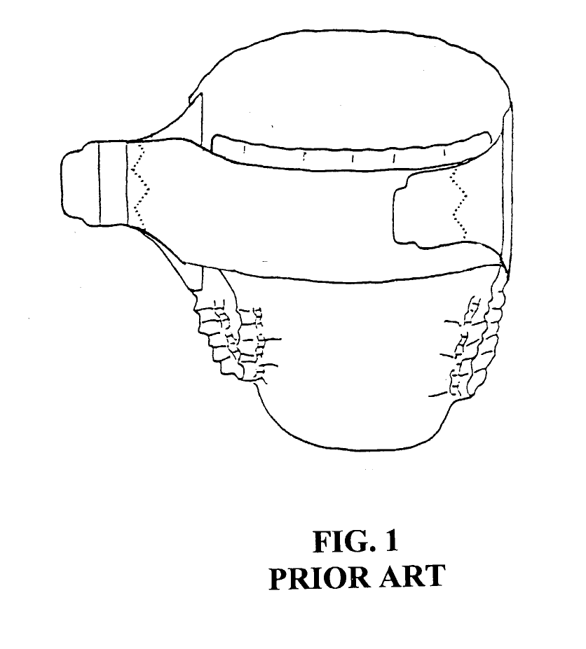

Five samples (Samples 6-10) of the previously-described configuration for a prefastened article as shown in FIG. 1 were obtained. The conventional disposable absorbent articles included a non-extensible attachment panel comprising a pattern unbonded material attached to a front waist region of the article. The attachment panel extended laterally across an outer surface of the front waist region and extended laterally outwardly from the front waist region to form laterally opposing front ear portions. The conventional disposable absorbent articles further included an extensible back ear panel bonded to laterally opposing edge portions of the back waist region. A fastening component was attached to each ear panel. Each of the front ear portions of the attachment panel were bonded to the corresponding extensible back ear panel at a laterally central portion of the back ear panel using passive bonds having a zig-zag bonding pattern. Thus, each conventional sample included a non-extensible attachment panel positioned on a front waist region that extended laterally therefrom and was bonded to a corresponding back ear panel that extended from a side portion of the back waist region. The back ear panel comprised a neck-bonded laminate material that was passively bonded to the attachment panel using 0.03 bonds having a zig-zag bonding pattern.

The five samples (Samples 1-5) of the diaper pant 20 of the present invention, each sample having side seams connecting a front waist region of the diaper pant 20 including the extensible outer cover and the extensible bodyside liner to a back waist region of the diaper pant 20 including the extensible outer cover and the extensible bodyside liner, were compared to the five samples (Samples 6-10) of the absorbent article.

The following measurements were taken for each Sample 1 through 5, as shown in Table 1, and each conventional Sample 6 through 10, as shown in Table 2, below. For Samples 1 through 5, the Initial Waist Measurement and the Final Waist Measurement were measured from side seam to opposing side seam. For Samples 6 through 10, the Initial Waist Measurement and Final Waist Measurement were measured from the passive bond pattern connecting the attachment panel to a central portion of the first back ear panel to the laterally opposing passive bond pattern connecting the attachment panel to a central portion of the second back ear panel.

The “Initial Waist Measurement” is a measurement of the back waist region between laterally opposing side seams (present invention) or the laterally opposing passive bond patterns (conventional absorbent article) when the back waist region is directly contacting the front waist region, without stretching the chassis.

The “Final Waist Measurement” is a measurement of the back waist region between laterally opposing side seams (present invention) or the laterally opposing passive bond patterns (conventional absorbent article) when the diaper pant or the absorbent article is positioned on a Step 4 baby model. A Step 4 baby model has a waist circumference that simulates a waist circumference of a baby weighing about 22 pounds to about 37 pounds.

The “Percent Extension” is calculated by dividing the difference between the Final Waist Measurement and the Initial Waist Measurement by the Initial Waist Measurement and multiplying the result by 100.

Referring to the measurements shown in Tables 1 and 2, the average Initial Waist Measurement of the diaper pant 20 of the present invention is similar to the average Initial Waist Measurement of the conventional absorbent article (128.60 mm and 128.0 mm, respectively). However, the average Final Waist Measurement of the diaper pant 20 was significantly greater than the average Final Waist Measurement of the conventional absorbent article (215.00 mm and 131.80 mm, respectively). As a result of the large disparity in extensibility, the average percent extension of the diaper pant 20 was significantly greater than the average percent extension of the conventional absorbent article (67% and 3% respectively).

TABLE 1
Percent Extension in Back Waist Region
(Diaper Pant of the Present Invention)

| | Initial Waist | Final Waist | Percent |
|---|---|---|---|
| | Measurement | Measurement | Extension |
| Sample # | (mm) | (mm) | (%) |
| 1 | 128 | 215 | 68 |
| 2 | 127 | 218 | 72 |
| 3 | 129 | 224 | 74 |
| 4 | 131 | 208 | 59 |
| 5 | 128 | 210 | 64 |
| Average | 128.60 | 215.00 | 67 |
| Stdv. | 1.52 | 6.40 | 6 |

TABLE 2
Percent Extension in Back Waist Region
(Conventional Absorbent Article)

| | Initial Waist | Final Waist | Percent |
|---|---|---|---|
| | Measurement | Measurement | Extension |
| Sample # | (mm) | (mm) | (%) |
| 6 | 128 | 134 | 5 |
| 7 | 127 | 129 | 2 |
| 8 | 124 | 126 | 2 |
| 9 | 130 | 134 | 3 |
| 10 | 131 | 136 | 4 |
| Average | 128.00 | 131.80 | 3 |
| Stdv. | 2.74 | 4.15 | 1 |

### EXAMPLE 2

One sample (Sample 1) of the diaper pant 20 of the present invention, as described in Example 1, having side seams connecting a front waist region of the diaper pant 20 including an extensible outer cover and an extensible bodyside liner 44 to a back waist region of the diaper pant 20 including the extensible outer cover and the extensible bodyside liner, were compared to ten samples (Samples 2-11) of the conventional absorbent article, as described in Example 1 above, each conventional sample having a non-extensible attachment panel positioned on a front waist region and extending laterally therefrom and bonded to a corresponding back ear panel bonded to a side portion of the back waist region. The back ear panel comprises a neck-bonded laminate material, which is passively bonded at a laterally central portion to the attachment panel using 0.03 bonds having a zig-zag bonding pattern.

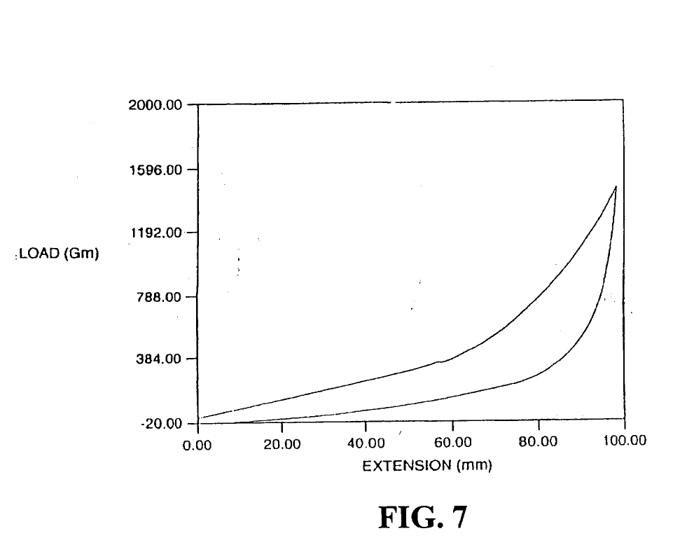

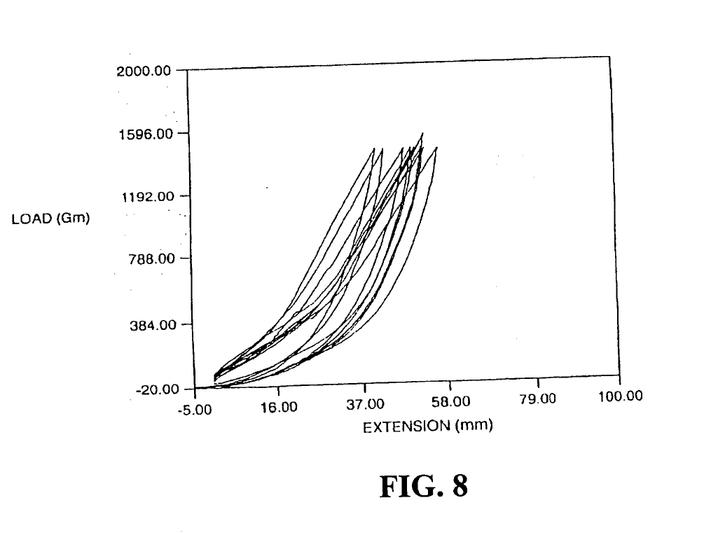

The following Tensile Tension measurements were taken for Sample 1, as shown in Table 3, and each conventional Sample 2 through 11, as shown in Table 4, below. For Samples 1 through 11, the Initial Waist Circumference Measurement and the Final Waist Circumference Measurement are measured. FIGS. 7 and 8 display several waist circumference measurements taken between the Initial Waist Circumference Measurement at 70 grams and the Final Waist Circumference Measurement at 1400 grams for Sample 1 and Samples 2-11, respectively.

The “Initial Waist Circumference Measurement” is a measurement of the circumference at the waist region of the diaper pant 20 or the absorbent article with the tension force set at 70 grams to represent a minimum tension force required for an absorbent article to remain positioned about the waist of a wearer.

The “Final Waist Circumference Measurement” is a measurement of the circumference at the waist region of the diaper pant 20 or the absorbent article with the tension force set at 1400 grams to represent a maximum comfortable tension force.

The “Fit Range” is the difference between the Final Waist Circumference Measurement and the Initial Waist Circumference Measurement.

Referring to the measurements shown in Tables 3 and 4, and corresponding FIGS. 7 and 8, the average Initial Waist Circumference Measurement of the diaper pant 20 of the present invention is similar to the average Initial Waist Circumference Measurement of the conventional absorbent article (420.4 mm and 422.6 mm, respectively). However, the average Final Waist Circumference Measurement of the diaper pant 20 was significantly greater than the average Final Waist Circumference Measurement of the conventionally absorbent article (592.9 mm and 515.2 mm, respectively). As a result of the large disparity in the extensibility, the average Fit Range of the diaper pant 20 was significantly greater than the average Fit Range of the conventional absorbent article (172.5 mm and 92.6 mm, respectively).

TABLE 3
Fit Range (Diaper Pant of the Present Invention)

| | Initial Waist | Final Waist | |
|---|---|---|---|
| | Circumference | Circumference | |
| | Measurement | Measurement | Fit Range |
| Sample # | (mm) | (mm) | (mm) |
| 1 | 420.4 | 592.9 | 172.5 |
| Average | 420.4 | 592.9 | 172.5 |
| Stdv. | 0.0 | 0.0 | 0.0 |

TABLE 4
Fit Range (Conventional Absorbent Article)

| | Initial Waist | Final Waist | |
|---|---|---|---|
| | Circumference | Circumference | |
| | Measurement | Measurement | Fit Range |
| Sample # | (mm) | (mm) | (mm) |
| 2 | 422.0 | 502.3 | 80.3 |
| 3 | 423.3 | 517.9 | 94.6 |
| 4 | 421.4 | 528.7 | 107.3 |
| 5 | 419.3 | 515.3 | 96 |
| 6 | 420.6 | 518.8 | 98.2 |
| 7 | 425.3 | 520.7 | 95.4 |
| 8 | 425.4 | 517.0 | 91.6 |
| 9 | 423.0 | 521.9 | 98.9 |
| 10 | 426.1 | 511.8 | 85.7 |
| 11 | 419.3 | 497.7 | 78.4 |
| Avg. | 422.6 | 515.2 | 92.6 |
| Stdv. | 2.5 | 9.2 | |

### EXAMPLE 3

Five samples of a disposable diaper pant 20 of the present invention were made by bonding a front edge portion of the diaper pant 20 including an extensible outer cover 42 and an extensible bodyside liner 44 to a back edge portion of the diaper pant 20 including the extensible outer cover 42 and the extensible bodyside liner 44 at laterally opposing side seams 68 to form a chassis 45 forming a waist opening 33. An extensible portion 62 of a fastener 60 was bonded between the front edge portion and the back edge portion at each side seam 68. The extensible portion 62 comprises a neck-bonded laminate material.

Five samples of a conventional disposable absorbent article, as described in Example 1 above, were obtained. The conventional disposable absorbent articles included a non-extensible attachment panel comprising a pattern unbonded material attached to a front waist region of the article. The attachment panel extended laterally across an outer surface of the front waist region and extended laterally outwardly from the front waist region to form laterally opposing front ear portions. The conventional disposable absorbent articles further included an extensible back ear panel bonded to laterally opposing edge portions of the back waist region. A fastening component was attached to each back ear panel. Each of the front ear portions of the attachment panel were bonded to the corresponding extensible back ear panel at a laterally central portion of the back ear panel using conventional passive bonds having a zig-zag bonding pattern.

The five samples of the diaper pant 20 of the present invention, each sample having side seams connecting a front waist region of the diaper pant 20 including an extensible outer cover and the extensible bodyside liner to a back waist region of the diaper pant 20 including the extensible outer cover and the extensible bodyside liner, were compared to the five samples of the conventional absorbent article, each conventional sample having a non-extensible attachment panel positioned on a front waist region and extending laterally therefrom and bonded to a corresponding back ear panel extending from a side portion of the back waist region. The back ear panel comprises a neck-bonded laminate material that is passively bonded to the attachment panel using 0.03 bonds having a zig-zag bonding pattern.

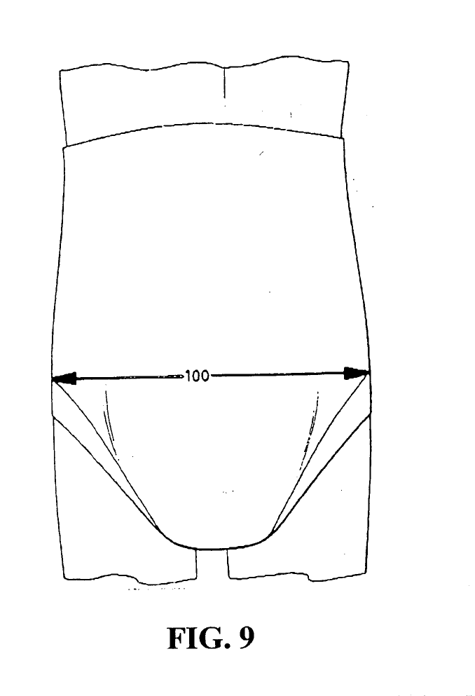

Each sample was placed on a Step 4 baby model to determine leg elastic curvature after initial application. A Step 4 baby model has a waist circumference that simulates a waist circumference of a baby weighing about 22 pounds to about 37 pounds. The following measurement was taken for each Sample 1 through 5 of the diaper pant of the present invention and the conventional article, as shown in Table 5, below. The Cross-Directional Measurement of the diaper pant or article was made as shown in FIG. 9.

Referring to the measurements shown in Table 5, the average Cross-Directional Measurement for the diaper pant 20 of the present invention was significantly greater than the average Cross-Directional Measurement of the conventional absorbent article (195.00 mm and 169.80 mm, respectively). As a result of the large disparity in extensibility, the average leg elastic curvature after initial application for the diaper pant 20 was significantly greater than the average leg elastic curvature after initial application of the conventional absorbent article.

The samples of the diaper pant of the present invention including straight leg elastic members, wherein the front waist region was passive bonded to the back waist region to join the facings resulted in better leg elastic curvature than did the conventional articles including highly curved leg elastic members and passive bonding to join the neck-bonded laminate material of the back ear panel to the pattern unbonded material of the attachment component. In other words, the top most effective leg elastic area in the back waist region of the present invention resulted in 25 mm larger cross-directional width measurement for the diaper pant of the present invention including straight leg elastic members, than the conventional article including highly curved leg elastic members.

TABLE 4
Leg Elastic Curvature of Percent Extension in Back Waist Region
(Diaper Pant of the Present Invention compared to Conventional
Absorbent Article)

| | Cross-Directional | Cross-Directional |
|---|---|---|
| | Measurement of | Measurement of Diaper |
| | Conventional Absorbent | Pant of Present Invention |
| Sample # | Article (mm) | (mm) |
| 1 | 175 | 196 |
| 2 | 166 | 198 |
| 3 | 168 | 192 |
| 4 | 168 | 193 |
| 5 | 172 | 196 |
| Average | 169.80 | 195.00 |
| Stdv. | 3.63 | 2.45 |

As shown in this Example, a prefastened diaper pant of the present invention having straight leg elastic members provides a better fit around the legs of the wearer than a conventional absorbent article having curved leg elastic members. Further, the manufacturing process is simplified by the use of straight leg elastic members, as used in the prefastened diaper pant of the present invention, rather than the use of curved leg elastic members used during the manufacturing process of the conventional absorbent article

While in the foregoing specification this invention has been described in relation to the preferred embodiments thereof, and many details have been set forth for purpose of illustration, it will be apparent to those skilled in the art that the invention is susceptible to additional embodiments and that certain of the details described herein can be varied considerably without departing from the basic principles of the invention.

What is claimed is:

1. A disposable diaper pant, comprising:
an extensible outer cover;
an extensible bodyside liner;
a chassis defined by the outer cover and the bodyside liner, the chassis having a front waist region, a back waist region and a crotch region intermediate the front waist region and the back waist region;
a first side seam bonding a first edge portion of the front waist region to a first edge portion of the back waist region;
a first fastener having an extensible portion positioned between and bonded to the first edge portion of the front waist region and the first edge portion of the back waist region;
a laterally opposing second side seam bonding a second edge portion of the front waist region to a second edge portion of the back waist region; and
a second fastener having an extensible portion positioned between and bonded to the second edge portion of the front waist region and the second edge portion of the back waist region,
wherein the chassis defines a waist opening, the chassis having a percent extension of at least about 40%.

2. The disposable diaper pant of claim 1 wherein the extensible outer cover is at least partially bonded to the extensible bodyside liner.

3. The disposable diaper pant of claim 1 further comprising an absorbent core positioned between the extensible outer cover and the extensible bodyside liner.

4. The disposable diaper pant of claim 1 wherein each of the first side seam and the second side seam extends from the waist opening to a corresponding leg opening.

5. The disposable diaper pant of claim 1 wherein each of the first side seam and the second side seam extends at least partially between the waist opening and a corresponding leg opening.

6. The disposable diaper pant of claim 1 wherein the extensible outer cover comprises a dual composite material.

7. The disposable diaper pant of claim 6 wherein the extensible outer cover comprises an extensible outer facing layer and an extensible inner facing layer.

8. The disposable diaper pant of claim 7 wherein the extensible outer facing layer comprises a spunbond nonwoven material.

9. The disposable diaper pant of claim 7 wherein the extensible inner facing layer comprises a film material.

10. The disposable diaper pant of claim 1 wherein the extensible portion of the first fastener and the extensible portion of the second fastener each comprises a neck-bonded laminate material.

11. The disposable diaper pant of claim 1 wherein the chassis has a percent extension of at least about 60%.

12. The disposable diaper pant of claim 1 wherein the chassis has a percent extension of at least about 70%.

13. The disposable diaper pant of claim 1 further comprising a first plurality of straight leg elastic members and a second plurality of straight leg elastic members, each extensible with the extensible outer cover to define a leg opening which conforms to a curvature of a wearer's corresponding leg.

14. A disposable absorbent article comprising:
an extensible outer cover;
an extensible bodyside liner;
an absorbent core positioned between the extensible outer cover and the extensible bodyside liner;
a chassis defined by the extensible outer cover, the absorbent core and the extensible bodyside liner, the chassis having a front waist region, a back waist region and a crotch region intermediate the front waist region and the back waist region; and
a first side seam bonding a first edge portion of the front waist region to a first edge portion of the back waist region and a laterally opposing second side seam bonding a second edge portion of the front waist region to a second edge portion of the back waist region, and forming a waist opening, a first leg opening and a second leg opening;
a first fastener having an extensible portion bonded with respect to the first side seam and a second fastener having an extensible portion bonded with respect to the second side seam, the extensible portion of each of the first fastener and the second fastener comprising a neck-bonded laminate material; and
an attachment panel positioned on an outer surface of the extensible outer cover, the first fastener and the second fastener having a fastening portion, each releasably engageable with the attachment panel.

15. The disposable absorbent article of claim 14 wherein the first side seam at least partially extends between the waist opening and the first leg opening and the second side seam extends at least partially between the waist opening and the second leg opening.

16. The disposable absorbent article of claim 14 further comprising a first plurality of straight leg elastic members extensible with the extensible outer cover to form the first leg opening, which conforms to a curvature of a wearer's corresponding first leg, and a second plurality of straight leg elastic members extensible with the extensible outer cover to form the second leg opening, which conforms to a curvature of the wearer's corresponding second leg.

17. The disposable absorbent article of claim 14 wherein the chassis having a percent extension of at least about 50% at the waist opening.

18. The disposable absorbent article of claim 14 wherein the attachment panel extends laterally across a portion of the one of the front waist region and the back waist region of the absorbent article.

19. The disposable absorbent article of claim 14 wherein the attachment panel extends laterally across a portion of the back waist region of the absorbent article.

20. The disposable absorbent article of claim 14 wherein the attachment panel comprises a pattern unbonded material.

21. An absorbent article comprising:
an outer cover extensible in the lateral direction of the absorbent article;
a bodyside liner extensible in the lateral direction and at least partially bonded to the outer cover;
an absorbent core positioned between the outer cover and the body side liner;
an extensible front waist region comprising a first portion of the outer cover, the front waist region having a first front ear portion and a laterally opposing second front ear portion;
an extensible back waist region comprising a second portion of the outer cover, the back waist region having a first back ear portion and a laterally opposing second back ear portion;
an extensible first fastener positioned between the first front ear portion and the first back ear portion;
a first side seam bonding the outer cover of the first front ear portion, the extensible first fastener and the outer cover of the first back ear portion;
an extensible second fastener positioned between the second front ear portion and the second back ear portion; and
a second side seam laterally opposing the first side seam and bonding the outer cover of the second front ear portion, the extensible second fastener and the outer cover of the second back ear portion,
wherein the outer cover, bodyside liner and the absorbent core defining a chassis forming a waist opening and having a percent extension of at least about 50% at the waist opening.

23. The absorbent article of claim 21 wherein each of the first fastener and the second fastener is refastenably engageable with respect to the front waist region.

24. The absorbent article of claim 21 wherein the outer cover comprises an extensible outer facing layer and an extensible inner facing layer laminated to the extensible outer facing layer.

25. The absorbent article of claim 24 wherein the extensible outer facing layer comprises a spunbond nonwoven material and the extensible inner facing layer comprises a film material.
